(19) United States
(12) Patent Application Publication
(10) Pub. No.: US 2026/0363553 A1
(43) Pub. Date: Nov. 19, 2026

(54) MONITORING SYSTEM, MONITORING METHOD, AND PROGRAM

(71) Applicant: SONY GROUP CORPORATION, TOKYO (JP)

(72) Inventors: TAKANORI FUKAZAWA, TOKYO (JP); NANA KAWAMURA, TOKYO (JP); HIROYASU BABA, TOKYO (JP)

(21) Appl. No.: 19/155,287

(22) PCT Filed: Feb. 13, 2024
(86) PCT No.: PCT/JP2024/004782

§ 371 Date: Aug. 11, 2025

(30) Foreign Application Priority Data
Mar. 2, 2023 (JP) 2023-031825

(51) Int. Cl. A61B 5/00; A61B 5/08

(52) U.S. Cl. CPC A61B 5/0082; A61B 5/0816; A61B 5/7203; A61B 5/7278; A61B 2576/00

(57) ABSTRACT

The present technology relates to a monitoring system, a monitoring method, and a program that enable respiratory monitoring with high robustness to be achieved. A monitoring system according to one aspect of the present technology acquires an RGB image obtained by capturing an image of a patient and a Depth image indicating Depth values relative to certain locations on the patient; tracks, based on the RGB image, a measurement region set in a respiration region of the patient; and when a tracking error of the measurement region occurs, resets the measurement region, based on the Depth image. The present technology can be applied to a system that performs monitoring of the respiratory condition of the patient in a non-contact manner.

### TECHNICAL FIELD

The present technology relates to a monitoring system, a monitoring method, and a program, and more particularly, relates to a monitoring system, a monitoring method, and a program that enable respiratory monitoring with high robustness to be achieved.

### BACKGROUND ART

Capnometers are commonly used for respiratory monitoring in clinical sites such as hospitals. The capnometer is a contact-type sensor that detects the concentration of carbon dioxide in breath. Because the capnometer is a contact-type sensor, measures are required to deal with false detection or undetectable state caused by poor contact.

Various non-contact respiratory monitoring systems have been proposed. One of the non-contact respiratory monitoring systems is a system using a camera (PTL 1).

### CITATION LIST

### Patent Literatures

### PTL 1

JP 2014-171574A

### SUMMARY

### Technical Problem

In order to apply the technology as described in PTL 1 to clinical practice, high robustness is required. In clinical practice, disturbances to respiratory monitoring are elicited due to movement of a patient, i.e., a subject, or a device, and due to changes in environmental light.

The present technology has been made in view of such circumstances, and makes it possible to achieve respiratory monitoring ensured with high robustness.

### Solution to Problem

A monitoring system according to one aspect of the present technology includes: an image processing unit that acquires an RGB image obtained by capturing an image of a patient and a Depth image indicating Depth values relative to certain locations on the patient; and a measurement unit that tracks, based on the RGB image, a measurement region set in a respiration region of the patient, and, when a tracking error of the measurement region occurs, resets the measurement region, based on the Depth image.

In one aspect of the present technology, an RGB image obtained by capturing an image of a patient and a Depth image indicating Depth values relative to certain locations on the patient are acquired; and a measurement region set in a respiration region of the patient is tracked, based on the RGB image. In addition, when a tracking error of the measurement region occurs, the measurement region is reset based on the Depth image.

### BRIEF DESCRIPTION OF DRAWINGS

FIG. 1 is a diagram illustrating a configuration example of a respiratory monitoring system.

FIG. 2 is a diagram illustrating an example of an image captured by an RGB-D camera.

FIG. 3 illustrates examples of waveforms indicating image feature amounts of an ROI.

FIG. 4 illustrates examples of ROIs.

FIG. 5 is a diagram illustrating an example of tracking of an ROI.

FIG. 6 is a diagram illustrating another example of tracking of an ROI.

FIG. 7 is a block diagram illustrating a configuration example of the respiratory monitoring system.

FIG. 8 is a flowchart illustrating respiratory monitoring processing.

FIG. 9 illustrates an example of calculation of a frequency feature amount.

FIG. 10 illustrates an example of calculation of a respiration intensity.

FIG. 11 is a flowchart illustrating an automatic ROI search processing performed in step S7 of FIG. 8.

FIG. 12 illustrates an example of selection of an ROI using Depth values.

FIG. 13 illustrates examples of IDRs of ROIs in regions.

FIG. 14 illustrates an example of detection of a respiration region.

FIG. 14 illustrates an example of detection of a respiration region.

FIG. 16 illustrates examples of evaluation of respiration intensities.

FIG. 17 illustrates a flow of the automatic ROI search processing.

FIG. 18 illustrates another flow of the automatic ROI search processing.

FIG. 19 is a diagram illustrating another configuration example of the respiratory monitoring system.

FIG. 20 is a block diagram illustrating the other configuration example of the respiratory monitoring system.

FIG. 21 is a flowchart illustrating respiratory monitoring processing for initial setting of the ROI automatically performed.

FIG. 22 is a diagram illustrating an example of tracking of an ROI using template matching.

FIG. 23 is a block diagram of a configuration example of a computer.

### DESCRIPTION OF EMBODIMENTS

Embodiments for carrying out the present technique will be described hereinafter.

The descriptions will be given in the following order.
1. Overview of Respiratory Monitoring System
2. Detailed Configuration of Respiratory Monitoring System
3. Respiratory Monitoring Processing
4. Modification Examples

<<Overview of Respiratory Monitoring System>>

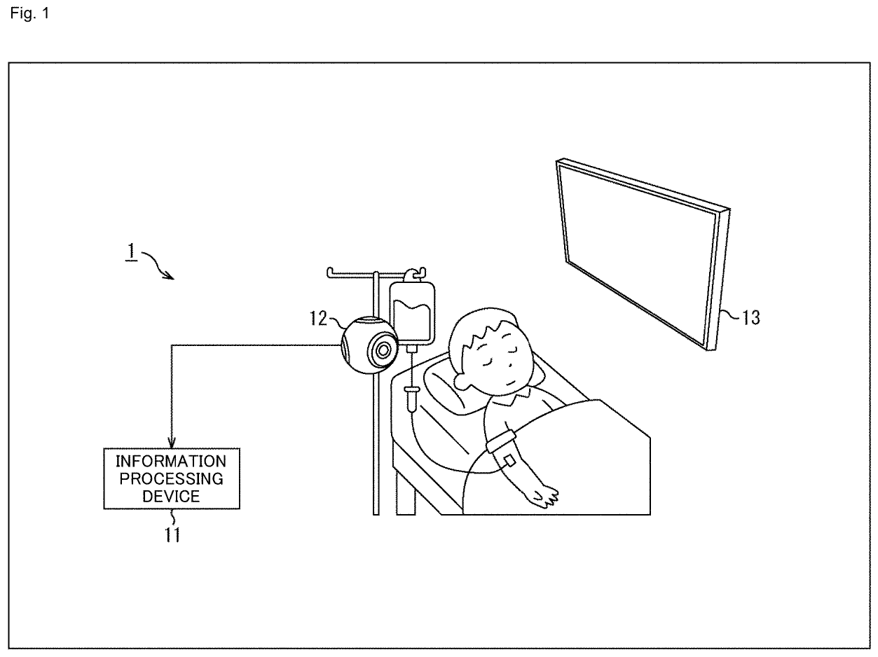

FIG. 1 is a diagram illustrating a configuration example of a respiratory monitoring system according to an embodiment of the present technology.

The respiratory monitoring system 1 in FIG. 1 has a configuration such that an RGB-D camera 12 that captures an image of a patient in an ICU or the like is connected to an information processing device 11 through wired or wireless communication. In the example of FIG. 1, the RGB-D camera 12 is attached to an intravenous stand placed next to a bed on which the patient is lying.

A monitor 13 is connected to the information processing device 11. The information processing device 11 and the monitor 13 are installed in, for example, an ICU. The information processing device 11 and the monitor 13 may be installed in another room of the medical facility, rather than in the ICU. The information processing device 11 and the monitor 13 may be installed in a same room, or may be installed in different rooms.

The respiratory monitoring system 1 is a system used by a medical staff member such as a doctor or a nurse as a user to monitor the respiratory condition of a patient. The respiratory condition is monitored based on images captured by the RGB-D camera 12. The monitor 13 displays a monitoring screen used by a medical staff member to check the respiratory condition of the patient, based on image signals transmitted from the information processing device 11 through wired or wireless communication.

In the example of FIG. 1, one RGB-D camera 12 that captures the appearance of one patient is connected to the information processing device 11. However, a plurality of cameras that capture images of different patients, respectively may also be connected to the information processing device 11.

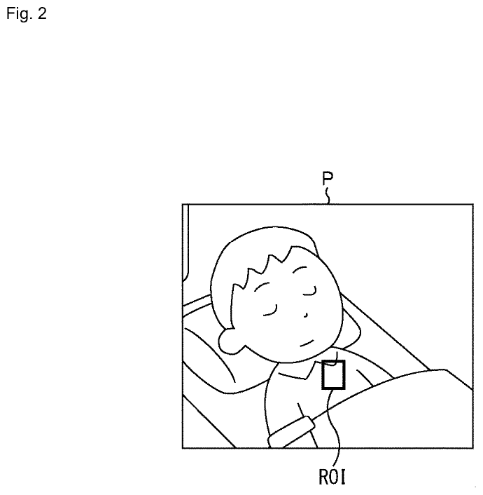

FIG. 2 is a diagram illustrating an example of an image captured by the RGB-D camera 12.

In an image captured by the RGB-D camera 12, a Region of Interest (ROI), which is a measurement region, is set as enclosed in a rectangular frame indicated. To observe the respiratory condition, such an ROI is set in a location where movement occurs due to respiration, such as the chest or abdomen. Although the shape of the ROI is rectangular, it may be another shape. A plurality of ROIs may be set. In the case where a plurality of ROIs are set, the sizes of the ROIs may be the same or different.

An RGB image and a Depth image are captured by the RGB-D camera 12. The RGB-D camera 12 is a camera equipped with a sensor for RGB images (visible light sensor) and a sensor for Depth images. The sensor for Depth images is made up of, for example, a Time of Flight (ToF) sensor.

The RGB image is an image having RGB values as the values of pixels. The Depth image is an image having Depth values that each indicate a distance to a subject, as the values of pixels. An ROI is set for each of the RGB image and the Depth image. ROIs may be set at corresponding locations on the respective images, or may be set in different locations.

Hereinafter, when there is no need to distinguish between the RGB image and the Depth image, which are captured by the RGB-D camera 12, they will be collectively referred to as the image.

Based on image data of an ROI in the entire image captured by the RGB-D camera 12, the information processing device 11 determines whether respiration of the patient is normal or abnormal.

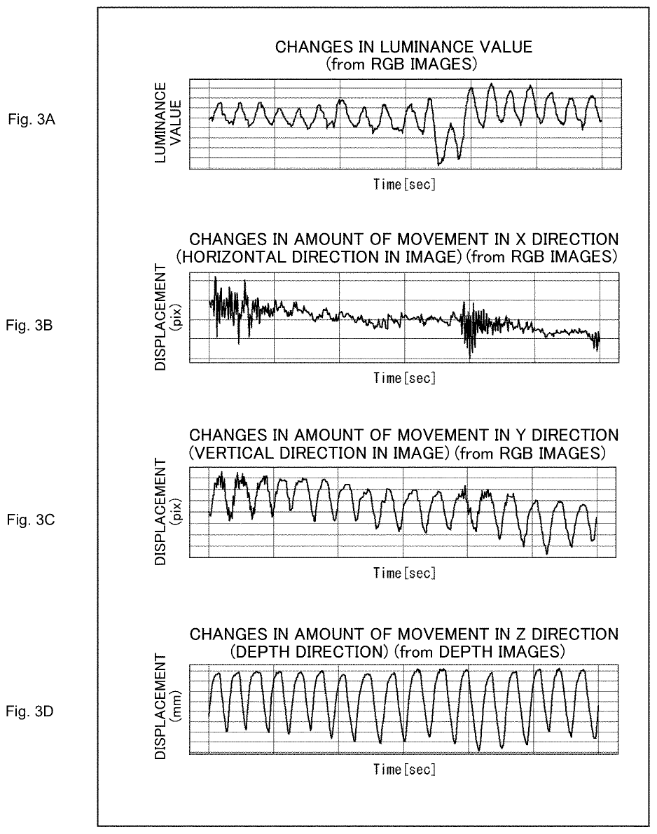

FIG. 3 illustrates examples of waveforms obtained by analyzing an ROI. The waveforms in FIG. 3 each indicate time series changes in image feature amount. Each image feature amount is a feature amount in the time domain that is determined from pixel data.

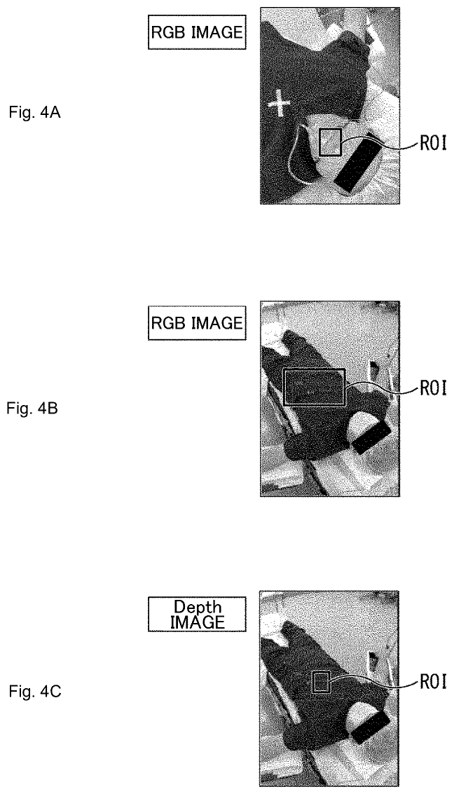

The waveform indicated in A of FIG. 3 is a waveform that indicates changes in luminance value. In A of FIG. 3, the horizontal axis represents time, and the vertical axis represents luminance value. The changes in luminance value are detected based on RGB images captured at certain times. For example, as illustrated in A of FIG. 4, an ROI is set in the location of a mask worn by a patient receiving oxygen therapy, and a waveform as indicated in A of FIG. 3 is generated based on an average of the luminance values of pixels that make up the ROI. As the mask becomes foggy in response to respiration, the luminance values of the ROI change.

The waveform indicated in B of FIG. 3 is a waveform that indicates changes in amount of movement in the X direction (horizontal direction in image). In B of FIG. 3, the horizontal axis represents time, and the vertical axis represents amount of displacement. The changes in amount of movement in the X direction is detected based on the RGB images captured at certain times. For example, as illustrated in B of FIG. 4, an ROI is set on the abdomen of a patient, and the movement of each feature point in the ROI in the X direction is estimated by using an Optical Flow method or the like. The estimation of the movement using the Optical Flow method is performed by calculating an amount of movement between frames of each of the feature points set in certain locations in the ROI.

The waveform indicated in C of FIG. 3 is a waveform that indicates changes in amount of movement in the Y direction (vertical direction in image). In C of FIG. 3, the horizontal axis represents time, and the vertical axis represents amount of displacement. The changes in amount of movement in the Y direction is also detected based on the RGB images captured at certain times. For example, similar to the amount of movement in the X direction, the movement in the Y direction of each feature point in the ROI illustrated in B of FIG. 4 is estimated.

When an image of the patient is captured with the head side being on the lower side and the feet side being on the upper side as illustrated in FIG. 4, for an ROI set on the chest and abdomen, the changes in amount of movement in the Y direction reflect the movement of the chest and abdomen due to respiration more than the changes in amount of movement in the X direction.

The waveform indicated in D of FIG. 3 is a waveform that indicates changes in amount of movement in the Z direction (depth direction of the image). In D of FIG. 3, the horizontal axis represents time, and the vertical axis represents amount of displacement. The changes in amount of movement in the Z direction is detected based on Depth images captured at certain times. For example, as illustrated in C of FIG. 4, an ROI is set on the abdomen of a patient, and a waveform as indicated in D of FIG. 3 is generated based on an average of the pixel values (Depth values) of pixels that make up the ROI. In the case where the ROI is set on the abdomen, the waveform obtained based on the Depth images reflects the movement of the abdomen due to respiration.

In this way, the respiratory monitoring system 1 generates respiratory waveform data based on the RGB image(s) and the Depth image(s), which are captured by the RGB-D camera 12, and performs respiratory monitoring in a non-contact manner. Respiratory monitoring based on both the RGB image(s) and the Depth image(s) may not always be performed, but an image to be used to perform respiratory monitoring may be selected depending on the situation.

During respiratory monitoring, the ROI is tracked so that the waveform for the same location can be continuously observed. When the patient moves, the location of the ROI changes according to the movement of the patient's body.

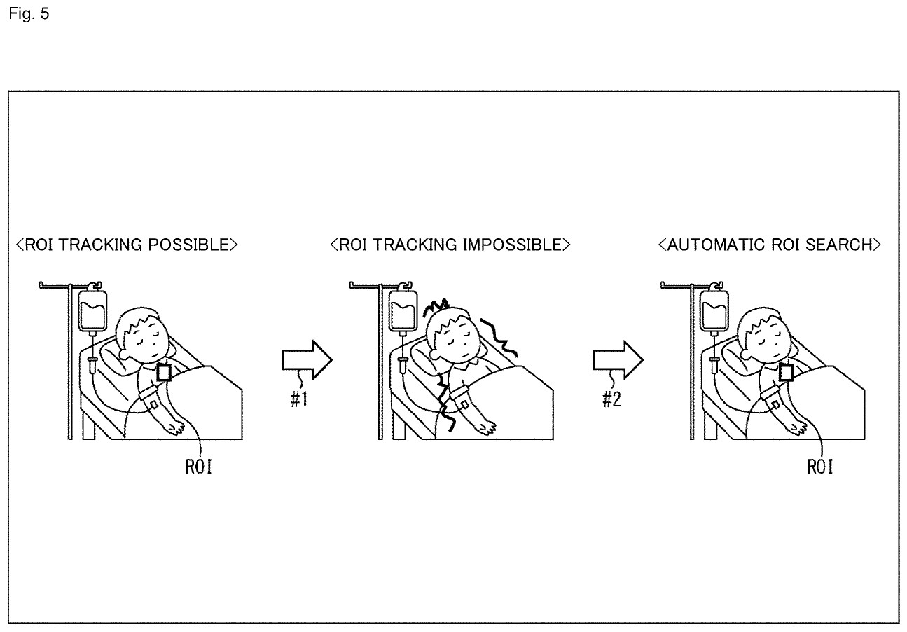

FIG. 5 is a diagram illustrating an example of tracking of an ROI. In FIG. 5, one ROI is set on the chest of a patient. For example, the initial location of the ROI is set manually by a nurse or the like. For ease of explanation, the RGB-D camera 12 is not illustrated from FIG. 5.

During respiratory monitoring, the ROI is tracked as illustrated at the leftmost in FIG. 5. For example, the ROI is tracked by analyzing the RGB image of each frame.

In this state, when the body of the patient moves significantly, for example as indicated by the head of arrow #1, the ROI cannot be tracked. If the movement is small, it is possible to track the ROI, but if the movement is large, such tracking of the ROI becomes impossible.

In the respiratory monitoring system 1, when a tracking error of the ROI occurs due to the movement of the patient, an automatic search for an ROI is performed as indicated by the head of arrow #2. The ROI is searched for by selecting a location where image data indicating the respiratory condition can be acquired, and the ROI found by the search is automatically set (reset). The respiratory monitoring continues based on the reset ROI. The automatic search for an ROI is performed, for example, by analyzing the Depth image(s).

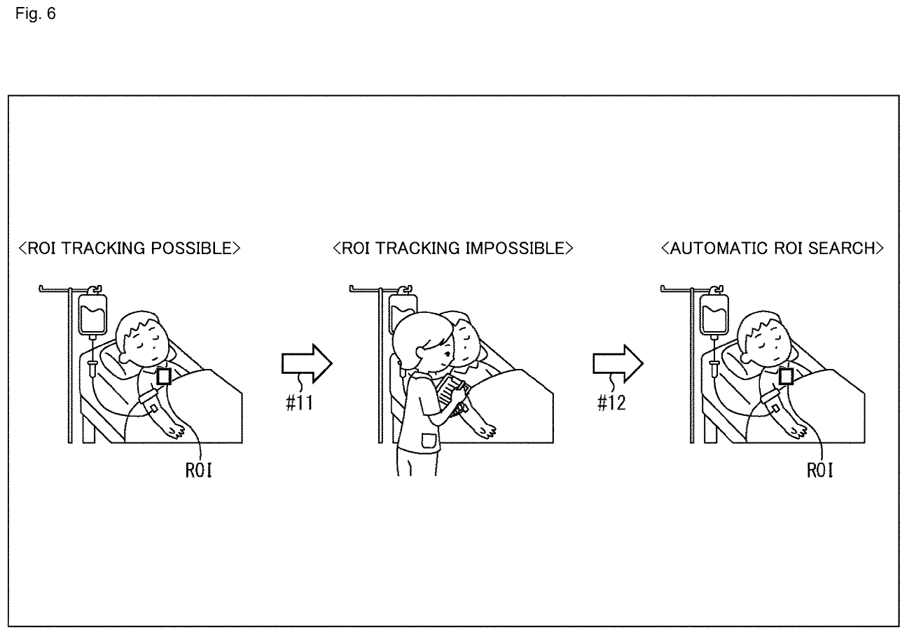

FIG. 6 is a diagram illustrating another example of tracking of an ROI.

As illustrated at the leftmost in FIG. 6, while the ROI is tracked by analyzing the RGB image(s), when a patient, which is the subject, is occluded by the intervention of a medical staff member, for example, as indicated by the head of arrow #11, the ROI cannot be tracked.

Even when a tracking error of the ROI occurs due to the patient being occluded, automatic search for an ROI is performed as indicated by the head of arrow #12, as in the case where a tracking error of the ROI occurs due to the movement of the patient.

In this way, the respiratory monitoring system 1 performs tracking of the ROI based on the RGB image(s), and when a tracking error occurs, automatic search for an ROI is performed based on the Depth image(s). Even when an ROI tracking error occurs, the ROI can be automatically reset to continue respiratory monitoring.

Since the RGB-D camera 12 is used for respiratory monitoring, it is possible to monitor the respiratory condition from a variety of perspectives based on the luminance values and the amounts of movement in the X, Y and Z directions. In addition, it is possible to improve the accuracy of abnormality detection based on such information from a variety of perspectives.

The details of processing for achieving an automatic ROI search function and a respiratory condition abnormality detection function will be described later with reference to flowcharts.

### <<Detailed Configuration of Respiratory Monitoring System>>

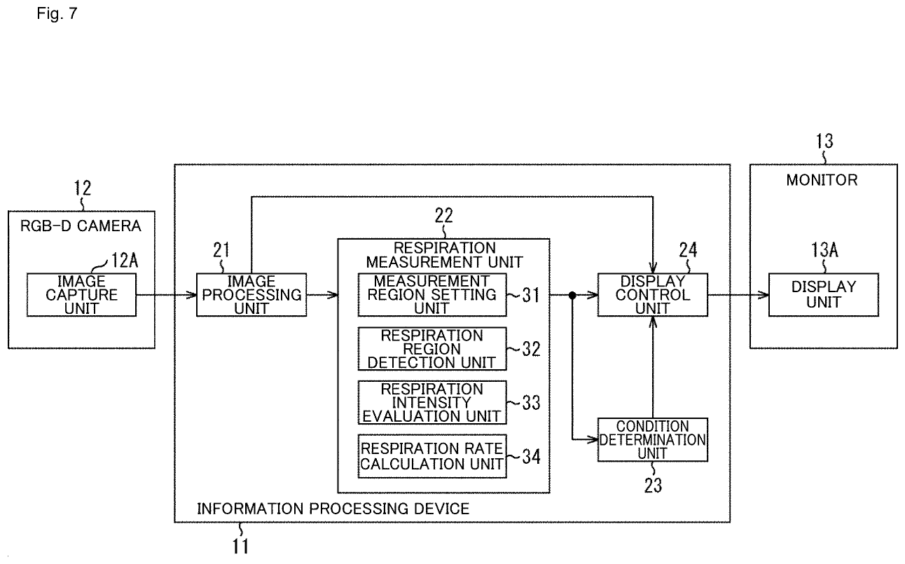

FIG. 7 is a block diagram illustrating a configuration example of the respiratory monitoring system 1.

As illustrated in FIG. 7, the information processing device 11 includes an image processing unit 21, a respiration measurement unit 22, a condition determination unit 23, and a display control unit 24. Other components such as an operation unit used for inputting information about a patient are also provided in the information processing device 11 as appropriate. The RGB image and the Depth image, which are captured by an image capture unit 12A of the RGB-D camera 12 are input to the image processing unit 21.

The image processing unit 21 of the information processing device 11 acquires the images transmitted from the RGB-D camera 12, and stores them in a memory. The image processing unit 21 outputs the RGB image to the display control unit 24, which causes the monitor 13 to display an image of a patient.

The image processing unit 21 also extracts image data of an ROI of each of the RGB image and the Depth image, and performs image processing on the resulting image data to analyze the image feature amounts of the ROIs. As described with reference to FIG. 3, respiratory waveform data indicating changes in luminance value and changes in amount of movement in each of the X, Y and Z directions is generated as analysis results.

The image processing unit 21 outputs the respiratory waveform data, which is the analysis results of the image feature amounts of the ROIs, to the respiration measurement unit 22. The RGB image and the Depth image are also supplied to the respiration measurement unit 22.

The respiration measurement unit 22 measures the respiratory condition of the patient based on the information supplied from the image processing unit 21. The respiration measurement unit 22 includes a measurement region setting unit 31, a respiration region detection unit 32, a respiration intensity evaluation unit 33, and a respiration rate calculation unit 34.

The measurement region setting unit 31 sets an ROI for respiratory monitoring. Information on the ROI set by the measurement region setting unit 31 is supplied to the image processing unit 21 and used for analysis of the ROI.

The respiration region detection unit 32 tracks the ROI based on the RGB image(s). The respiration region detection unit 32 also calculates a frequency feature amount based on the image feature amount of the ROI to detect a respiration region. The frequency feature amount is a feature amount in the frequency domain that is obtained by performing a predetermined calculation such as FFT on the image feature amount, which is a feature amount in the time domain.

The respiration intensity evaluation unit 33 evaluates the respiration intensity based on the frequency feature amount of the ROI.

The respiration rate calculation unit 34 calculates a respiration rate of the patient based on the frequency feature amount of the ROI.

The information obtained by the components of the respiration measurement unit 22 is supplied, as monitoring results of the respiratory condition, to the condition determination unit 23 and the display control unit 24. The details of processing of each component of the respiration measurement unit 22 will be described later.

Based on the information supplied from the respiration measurement unit 22, the condition determination unit 23 determines whether or not there is something abnormal with the respiration of the patient. Information indicating the result of the determination by the condition determination unit 23 is supplied to the display control unit 24.

The display control unit 24 controls the display of a monitoring screen that is a screen used for respiratory monitoring for the patient. For example, on the monitoring screen, displayed is information such as respiratory waveform and respiration rate along with patient images having frames which are each made up of an RGB image. When the condition determination unit 23 determines that the respiration of the patient is abnormal, the display control unit 24 causes information indicating abnormal to be displayed on the monitoring screen.

The image capture unit 12A of the RGB-D camera 12 captures constantly images of the patient during respiratory monitoring, and outputs the captured RGB images and Depth images to the information processing device 11.

The display unit 13A of the monitor 13 displays the monitoring screen under the control of the display control unit 24.

### <<Respiratory Monitoring Processing>>

### <Overall Flow>

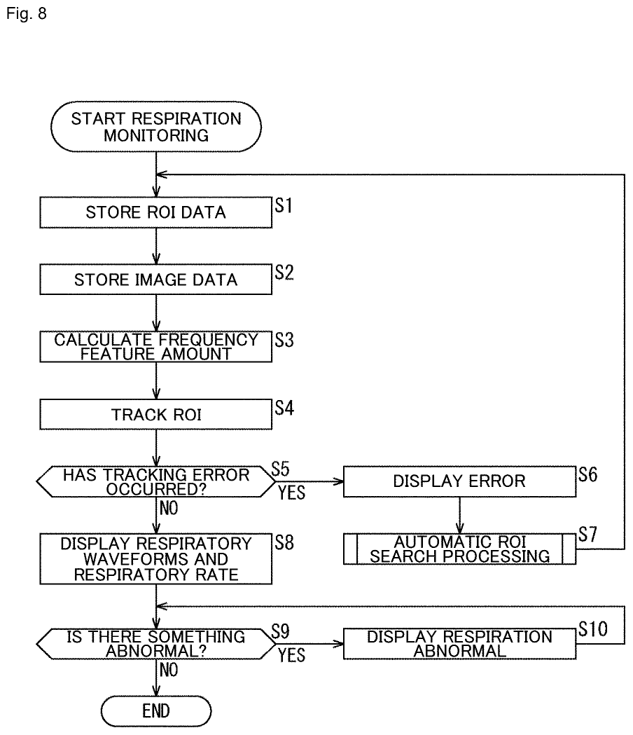

Processing of the respiratory monitoring system 1 having the above-described configuration will now be described with reference to a flowchart of FIG. 8. During respiratory monitoring, an RGB image and a Depth image obtained by capturing an image of a patient are supplied from the RGB-D camera 12 to the information processing device 11.

In step S1, the measurement region setting unit 31 of the respiration measurement unit 22 sets an ROI in each of the RGB image and the Depth image, and stores ROI data that is information indicating the location and range of each ROI. The initial setting of the ROI is performed manually by a medical staff member while viewing the RGB image, for example. On the monitor 13, a screen used for initial setting of the ROI is displayed.

In step S2, the image processing unit 21 acquires the images transmitted from the RGB-D camera 12, and stores their image data in the memory. The image processing unit 21 analyzes the ROI of each of the RGB image and the Depth image, and generates respiratory waveform data as image feature amounts. Respiratory waveform data as described with reference to FIG. 3 is generated based on the ROI of each of the RGB image and the Depth image.

In step S3, the respiration region detection unit 32 calculates a frequency feature amount of the ROI based on the respiratory waveform data.

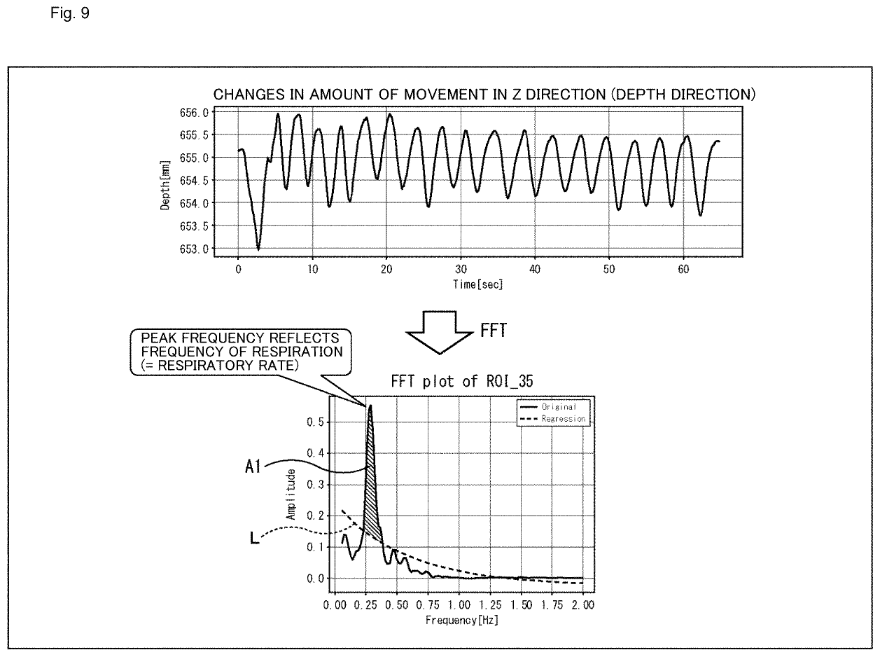

FIG. 9 illustrates an example of calculation of the frequency feature amount. On the upper side of FIG. 9, a waveform indicating changes in amount of movement in the Z direction is illustrated.

A calculation such as FFT is performed on the waveform data indicating changes in amount of movement in the Z direction, and a frequency feature amount having a peak at a predetermined frequency, as illustrated on the lower side of FIG. 9, is generated. In a graph illustrated on the lower side of FIG. 9, the horizontal axis indicates frequency, and the vertical axis indicates signal intensity. A curve Lis a regression curve determined based on the frequency feature amount. A hatched region A1 on the lower side of FIG. 9 is a region surrounded by the regression curve and an amplitude spectrum.

In the frequency feature amount thus obtained, the peak frequency reflects a frequency of respiration, that is, a respiration rate, as indicated in a balloon. The respiration rate calculation unit 34 calculates the respiration rate based on such a frequency feature amount generated by the respiration region detection unit 32.

The area of the region A1 reflects the respiration intensity. When the amplitude of a frequency around the peak frequency is relatively larger than the amplitude of other frequencies, the area of the region A1 becomes large. The respiration intensity evaluation unit 33 calculates the respiration intensity based on the frequency feature amount generated by the respiration region detection unit 32.

A calculation of a frequency feature amount using changes in amount of movement in the Z direction, which is the depth direction, has been described with reference to FIG. 9. However, the respiration rate and the respiration intensity can be determined in a similar manner by using changes in luminance value and changes in amount of movement in a planar direction (X direction, Y direction). The respiration rate and the respiration intensity may be obtained based on all four types of analysis results (ROI analysis results), or based on any one type of analysis result.

The respiration rate and the respiration intensity as monitoring results may be determined by combining the respiration rate and the respiration intensity obtained based on a plurality of types of analysis results, such as by averaging each of the respiration rates and the respiration intensities obtained based on two types of analysis results.

In this way, the respiration rate and the respiration intensity are obtained as monitoring results based on at least one of the changes in luminance value, the changes in amount of movement in the X direction, changes in amount of movement in the Y direction, and changes in amount of movement in the Z direction.

In step S4 of FIG. 8, the respiration region detection unit 32 tracks the ROI based on the image feature amount(s) of the RGB image. As described above, the ROI is tracked by calculating an amount of movement between frames of each of the feature points set in certain locations within the ROI.

In step S5, the respiration region detection unit 32 determines whether or not a tracking error of the ROI has occurred.

### Examples of Causes of Tracking Errors Include the Following

The patient or the RGB-D camera 12 moves significantly, making it impossible to track the ROI.
A medical staff member or someone appears in front of the RGB-D camera 12, making it impossible to capture an image of a region where the ROI is set.
The patient is covered with a blanket or something similar, occluding a region where the ROI is set.

The method of determining a tracking error is, for example, as follows.
A similarity of image feature amounts between ROIs and its surroundings is calculated for each frame or at regular time intervals, and if the similarity falls below a threshold value, it is determined that an error has occurred.

The tracking of the ROI may be performed based on the Depth image(s). In this case, for example, a distance to the subject is obtained for each frame or at regular time intervals, and if a change in the distance is beyond a threshold value, it is determined that a tracking error has occurred. For example, if the distance to the subject is 1 m in the initial setting of the ROI but changes to 2 m at a certain timing, it is determined that a tracking error has occurred.

In this way, it is possible to determine that a tracking error has occurred not only when the ROI cannot be tracked, but also when various conditions using image feature amounts are no longer satisfied.

If it is determined in step S5 that a tracking error has occurred, then in step S6, the respiration region detection unit 32 outputs to the display control unit 24 information indicating that a tracking error of the ROI has occurred. The display control unit 24 displays on the monitoring screen the information indicating that a tracking error has occurred, thereby displaying the error. On the monitoring screen, a message or icon indicating that a tracking error of the ROI has occurred is displayed.

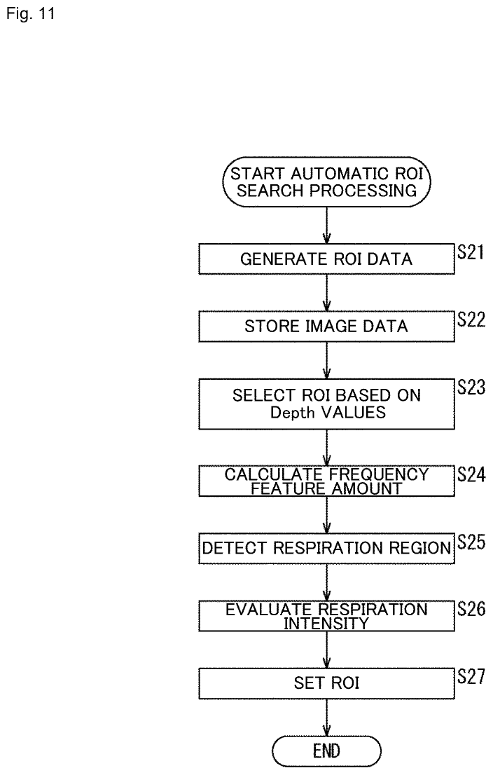

After the display of the tracking error of the ROI is started, automatic ROI search processing is performed in step S7. After an ROI is automatically reset by the automatic ROI search processing, the processing of step S1 and subsequent steps is repeated. The automatic ROI search processing performed in step S7 will be described later with reference to a flowchart of FIG. 11.

On the other hand, if it is determined in step S5 that no tracking error of the ROI has occurred, then in step S8, the display control unit 24 displays a respiratory waveform(s) and a respiration rate on the monitoring screen.

The respiratory waveform(s) are displayed based on the analysis results of the ROI generated by the image processing unit 21, for example. All of the respiratory waveforms, including a respiratory waveform that indicates changes in luminance value and respiratory waveforms that indicate changes in amount of movement in the X, Y, and Z directions, may be displayed, or at least one of the respiratory waveforms may be displayed. A waveform generated by combining a plurality of waveforms may be displayed.

In step S9, the condition determination unit 23 performs abnormal respiration determination. The abnormal respiration determination is processing of determining whether or not there is something abnormal with the respiratory condition of the patient.

For example, the condition determination unit 23 performs pre-processing on the analysis results of the ROI, and then performs the abnormal respiration determination. The pre-processing is performed, for example, to unify the units of the analysis results. Using the RGB-D camera 12, which makes it possible to detect respiratory movement using information from a variety of perspectives, such as the luminance values and the amounts of movement in the X, Y and Z directions, requires preprocessing for information in different units.

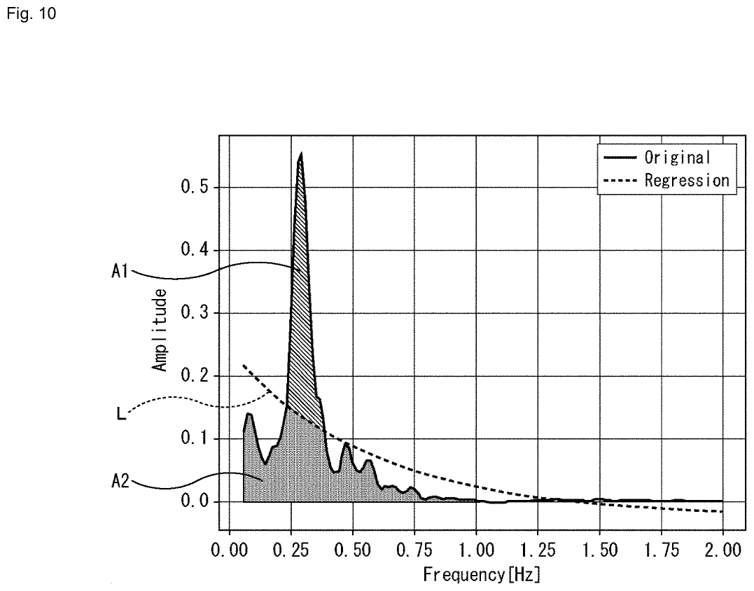

The pre-processing is performed as follows.
Standardization of numerical data.
If there is a misalignment between the angle of view of the RGB image and the angle of view of the Depth image, calibration is performed to convert the camera coordinate system (unit: pixel) to a world coordinate system (unit: mm). This makes it possible to handle the amount of movement in each of the X, Y and Z directions in the same unit.
The ratio of the area of a region A1 to the area of a region A2 illustrated in FIG. 10 is calculated as the respiration intensity. The region A1 is a region surrounded by a regression curve and an amplitude spectrum for data the frequency domain. The region A2 is a region of an amplitude spectrum below the regression curve.

As the abnormal respiration determination, threshold processing based on data of the analysis results obtained by such pre-processing, or processing using a model generated by machine learning is performed.

In the abnormal respiration determination using the threshold processing, for example, if the respiration rate or the respiration intensity is below a threshold value for a certain period of time, it is determined that there is something abnormal with the respiratory condition of the patient.

On the other hand, in the abnormal respiration determination using the model generated by machine learning, data such as respiratory waveforms and a frequency feature amount is input into the model, and based on the output, it is determined whether or not there is something abnormal with the respiratory condition of the patient. A model is prepared in advance in the condition determination unit 23, which receives, as inputs, respiratory waveforms, a frequency feature amount, and others and outputs a result of classification into a respiration pattern. If the result of classification into the respiration pattern output by the model indicates an abnormal pattern, it is determined that there is something abnormal with the respiratory condition of the patient.

Returning to the explanation of FIG. 8, if it is determined in step S9 that there is something abnormal with the respiratory condition of the patient, then in step S10, the display control unit 24 displays, on the monitoring screen, information indicating that there is something abnormal with the respiration of the patient, thereby displaying respiration abnormal. On the monitoring screen, a message or icon indicating that there is something abnormal with the respiration of the patient is displayed.

On the other hand, if it is determined in step S9 that there is nothing abnormal with the respiratory condition of the patient, the processing of FIG. 8 ends. The above processing is repeated during the respiratory monitoring.

### <Automatic ROI Search Processing>

Automatic ROI search processing performed in step S7 of FIG. 8 will now be described with reference to the flowchart in FIG. 11. The automatic ROI search processing is performed based on, for example, a Depth image.

In step S21, the measurement region setting unit 31 generates a plurality of ROIs, and generates ROI data indicating the location and range of each ROI. The ROIs thus generated are candidates for an ROI that will be finally set. For example, the entire Depth image is divided into a grid, and each of the regions thus divided is generated as a candidate for the ROI.

In step S22, the image processing unit 21 acquires the Depth image transmitted from the RGB-D camera 12, and stores its image data in the memory. The image processing unit 21 analyzes each ROI in the Depth image, and calculates changes in amount of movement in the Z direction as an image feature amount.

In step S23, the respiration region detection unit 32 selects an ROI based on the corresponding Depth values.

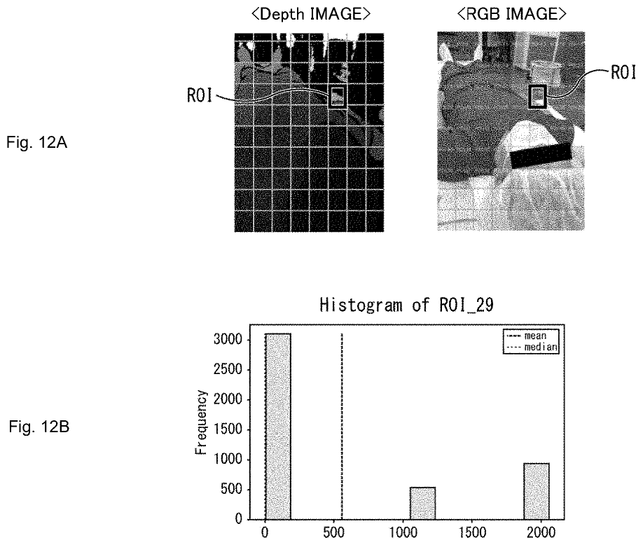

FIG. 12 illustrates an example of selection of an ROI using Depth values.

A Depth image illustrated on the left side of A of FIG. 12 is a Depth image acquired when a subject appearing in an RGB image illustrated on the right side is the target. Each grid-like region is set on the Depth image and generated as a candidate for the ROI. B of FIG. 12 indicates a histogram of Depth values of the ROI set at the location indicated in A of FIG. 12.

The selection of an ROI by the respiration region detection unit 32 is performed based on such a histogram. The Depth values of ROIs set at a boundary between the background and the patient vary widely. For example, the respiration region detection unit 32 excludes ROIs, each of which has larger variation in Depth values than a threshold value, to perform the selection of an ROI. The variation in Depth values is expressed, for example, by a coefficient of variation as the following Equation (1).

$$[ Math . 1 ]$$

$$Coefficient of variation = \frac{Standard deviation}{Average} \qquad ( 1 )$$

The respiration region detection unit 32 also calculates an Invalid Depth Ratio (IDR) for each ROI, which indicates the ratio of pixels with invalid Depth values, and excludes ROIs, each of which has a larger IDR than a threshold value, to perform the selection of an ROI. The IDR is expressed by the following Equation (2).

$$[ Math . 2 ]$$

$$IDR ( Invalid Depth Ratio ) = \frac{{N}_{invalid}}{{N}_{total}} \qquad ( 2 )$$

N_{invalid} is the number of pixels having an invalid Depth value. Nₜₒₜₐₗ is the number of pixels making up an ROI. For example, a pixel having a Depth value of 0 (distance detection failed) or a pixel having a Depth value outside a valid distance range is a pixel having an invalid Depth value. The valid distance range is set to, for example, 10 cm or more and 200 cm or less.

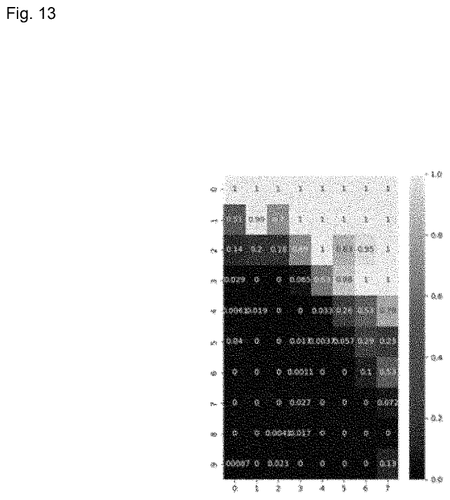

The IDR of each ROI illustrated in FIG. 12 is obtained as illustrated in FIG. 13. The IDR of ROIs set in the background or in the boundary between the patient and the background is high.

ROIs, each of which has an average of Depth values not within a valid range, may be simply excluded to perform the selection of an ROI. As the valid range, a predetermined distance is set such as within 200 cm.

In this way, the selection of an ROI by the respiration region detection unit 32 is performed based on the Depth values indicated by the Depth image, by excluding at least one of: regions where a ratio of pixels having undetected Depth values is greater than a threshold value, regions where a distance to the subject is outside a predetermined range, and regions where a coefficient of variation of the Depth values is greater than a threshold value.

In step S24 of FIG. 11, the respiration region detection unit 32 calculates a frequency feature amount of the ROI selected in step S23. If a plurality of ROIs are selected, the frequency feature amount of each ROI is calculated. By performing a calculation such as FFT on waveform data indicating changes in amount of movement in the Z direction, a frequency feature amount such as that described with reference to FIG. 9 is generated.

In step S25, the respiration region detection unit 32 detects an ROI that is to be a respiration region based on the frequency feature amount. The ROI to be a respiration region is detected, for example, using a coefficient of determination R² between the amplitude spectrum of the waveform indicating the changes in amount of movement in the Z direction and a regression curve.

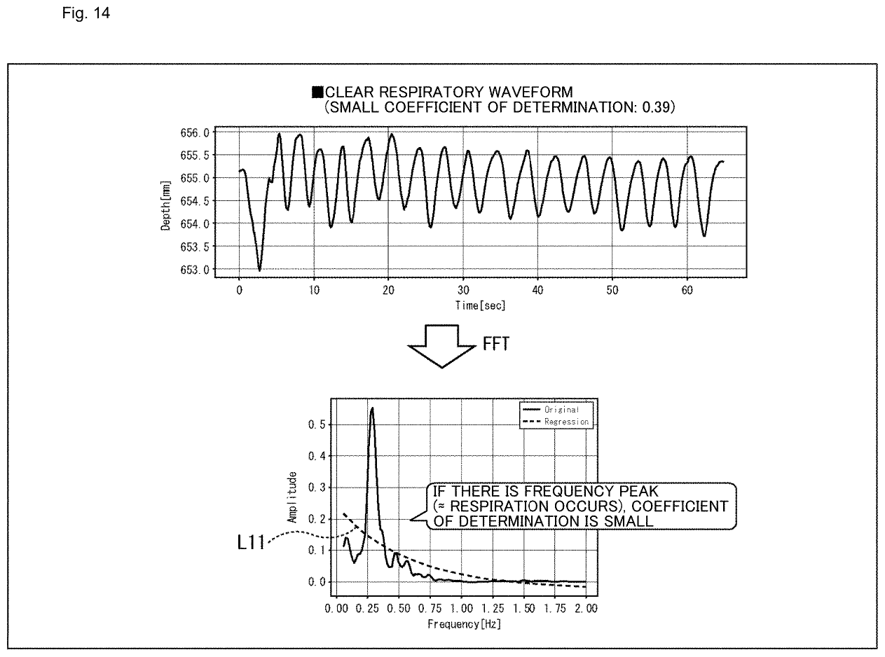

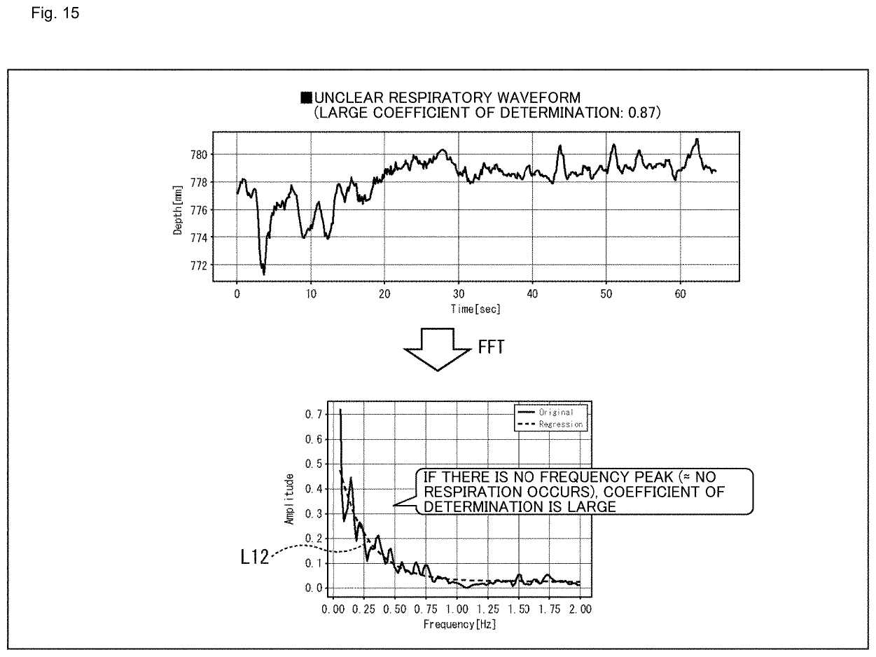

FIGS. 14 and 15 illustrate examples of detection of a respiration region.

FIG. 14 illustrates an example of detection in which the periodicity of the respiratory waveform is clear. When a calculation such as FFT is performed on such waveform data, a frequency feature amount in which a peak appears at a predetermined frequency, as illustrated on the lower side of FIG. 14, is obtained.

A peak appearing in the amplitude values means that a movement corresponding to respiration occurs in the ROI. When a peak appears at a certain frequency, amplitude values around the peak frequency deviate from the values of the regression curve (curve L11), resulting in a small coefficient of determination R². In the example of FIG. 14, a coefficient of determination R² of 0.39 is obtained.

On the other hand, FIG. 15 illustrates an example of detection in which the periodicity of the respiratory waveform is unclear. When a calculation such as FFT is performed on such waveform data, a frequency feature amount in which no clear peak in the amplitude values appears, as illustrated on the lower side of FIG. 15, is obtained.

No clear peak appearing in the amplitude values means that a movement corresponding to respiration does not occur in the ROI. When no peak appears, the amplitude value at each frequency becomes close to the value of the regression curve (curve L12), resulting in a large coefficient of determination R². In the example of FIG. 15, a coefficient of determination R² of 0.87 is obtained.

For example, an ROI, having a frequency feature amount as illustrated in FIG. 14, in which a coefficient of determination R² is smaller than a threshold value, is detected as a respiration region. For example, a plurality of ROIs are detected as respiration regions.

In step S26 of FIG. 11, the respiration intensity evaluation unit 33 evaluates the respiration intensity of each respiration region detected in step S25. The evaluation of the respiration intensity is based on the area of an amplitude spectrum around the respiration frequency.

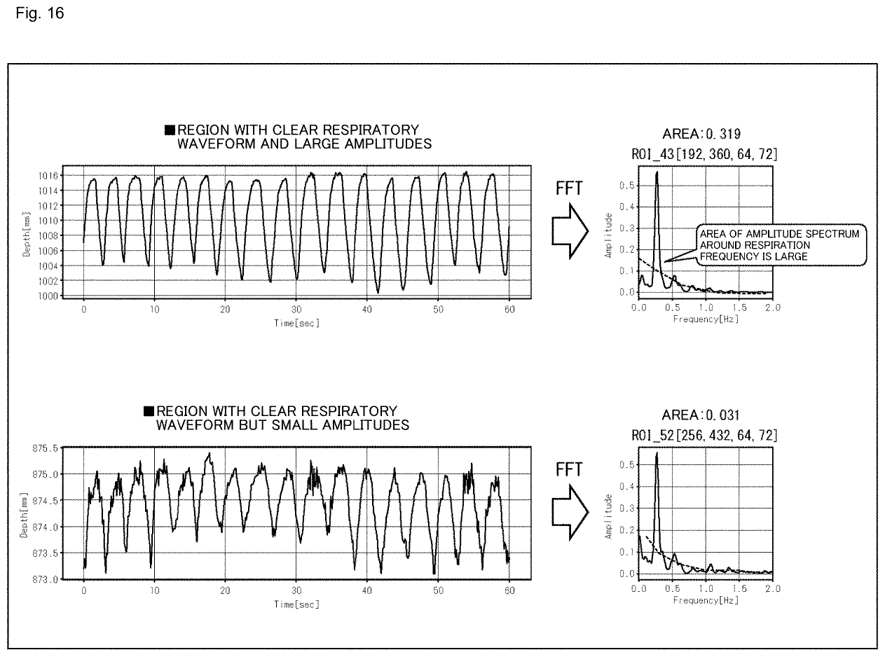

FIG. 16 illustrates examples of evaluation of respiration intensities.

A waveform on the upper side of FIG. 16 is a waveform with clear periodicity and large amplitudes, while a waveform on the lower side is a waveform with clear periodicity but small amplitudes.

As illustrated on the right side of FIG. 16, the area of an amplitude spectrum around the respiration frequency is calculated based on the frequency feature amount to evaluate the respiration intensity. In the examples of FIG. 16, the area of an amplitude spectrum around the respiration frequency is calculated as 0.319 for a waveform on the upper side, and 0.031 for a waveform on the lower side. The ROI having the frequency feature amount indicated on the upper side of FIG. 16 has a higher respiration intensity than the ROI having the frequency feature amount indicated on the lower side.

In step S27 of FIG. 11, the measurement region setting unit 31 selects and sets, for example, the ROI having the highest respiration intensity. The ROI may be selected by another algorithm using respiration intensities rather than the ROI having the highest respiration intensity. A plurality of ROIs may be selected and set as one ROI.

Through the above-described processing, in response to the occurrence of a tracking error of the ROI, an ROI is automatically reset. Thereafter, the processing returns to step S7 of FIG. 8 to perform the processing of step S1 and subsequent steps using the automatically reset ROI.

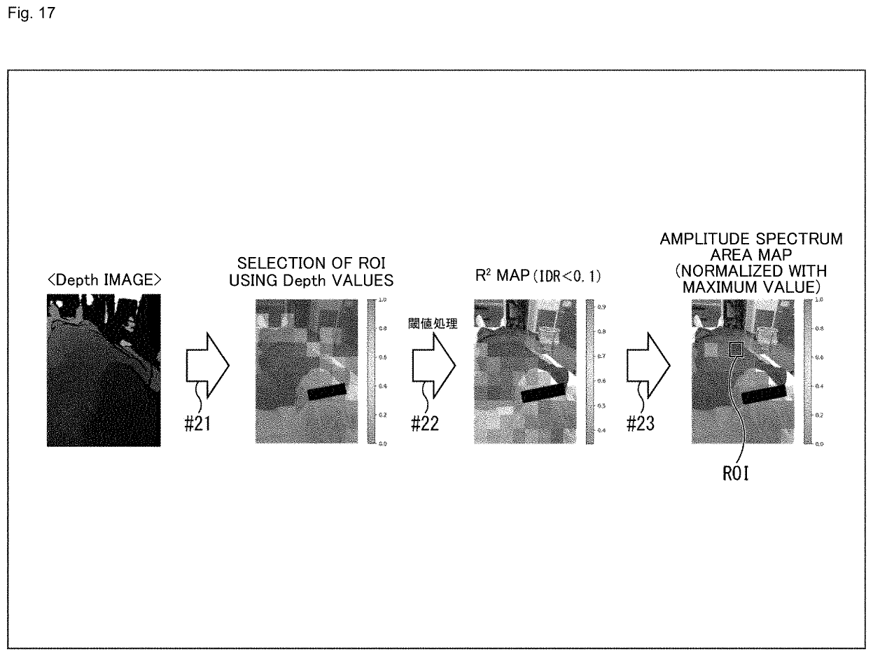

FIG. 17 illustrates a flow of the automatic ROI search processing as described above.

As indicated by the head of arrow #21, selection of an ROI is performed using Depth values (step S23). In addition, an R² map, which is a map of the coefficient of determination R² of each ROI selected by threshold processing using the Depth values, is generated as indicated by the head of arrow #22 (step S25).

As indicated by the head of arrow #23, a respiration intensity of a respiration region detected as an ROI having a coefficient of determination R² smaller than the threshold value is calculated based on the area of an amplitude spectrum (step S26).

An amplitude spectrum area map illustrated at the rightmost in FIG. 17 is a map indicating the area of the amplitude spectrum of each respiration region, that is, the respiration intensity. In an example of FIG. 17, an abdominal region, where the respiration intensity is the highest, is finally set as the ROI.

In this way, even if the ROI cannot be tracked due to disturbances that occur in clinical practice, an ROI can be automatically re-set, making it possible to monitor the respiratory condition of the patient stably and with high accuracy. It is also possible to provide safer respiratory care for the patient.

In addition, by performing respiratory monitoring based on information from a variety of perspectives, such as tracking the ROI based on an RGB image(s) and automatically searching for an ROI based on a Depth image(s), it is possible to achieve respiratory monitoring with high robustness.

In the above description, the automatic ROI search processing is performed using only the Depth image(s). However, the RGB image(s) may be used for at least a part of processing making up the automatic ROI search processing.

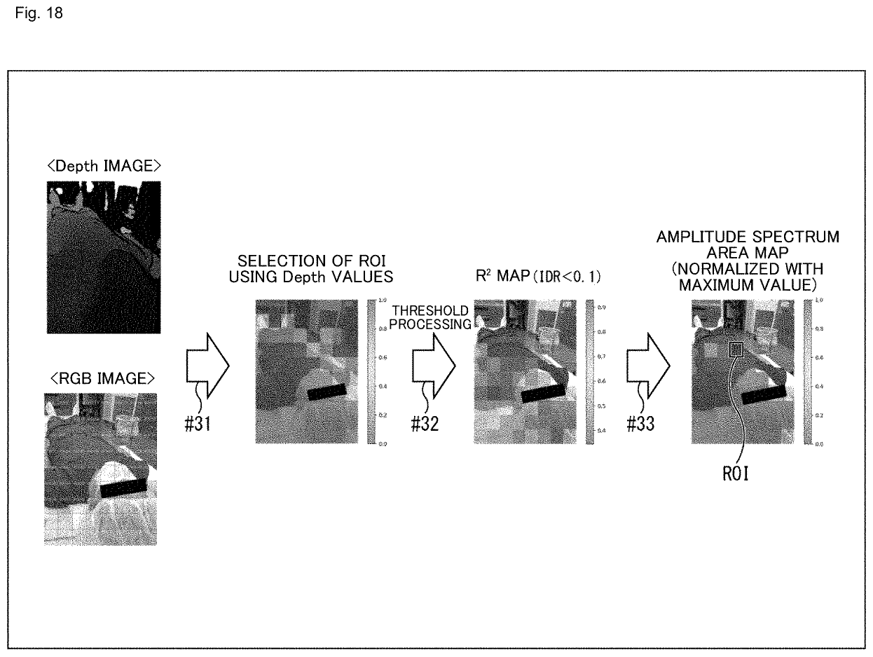

FIG. 18 illustrates another flow of the automatic ROI search processing.

As indicated by the head of arrow #31, selection of an ROI is performed using Depth values. In addition, an R² map, which is a map of the coefficient of determination R² of each ROI selected by threshold processing using the Depth values, is generated as indicated by the head of arrow #32.

In this example, changes in luminance values and changes in amounts of movement in the X and Y directions are determined based on RGB images, and an R² map is generated based on a frequency feature amount. An R² map may be generated by combining changes in luminance values, changes in amounts of movement in the X and Y directions, which are determined based on RGB images, and changes in amount of movement in the Z direction, which is determined based on Depth images.

As indicated by the head of arrow #23, a respiration intensity of a respiration region detected as an ROI having a coefficient of determination R² smaller than the threshold value is calculated based on the area of an amplitude spectrum. A respiration intensity is also calculated using the frequency feature amounts of the RGB images as appropriate.

By using RGB images and Depth images for the automatic search for an ROI, it is possible to set the ROI more accurately based on information from a variety of perspectives.

### Modification Examples

### <System Configuration>

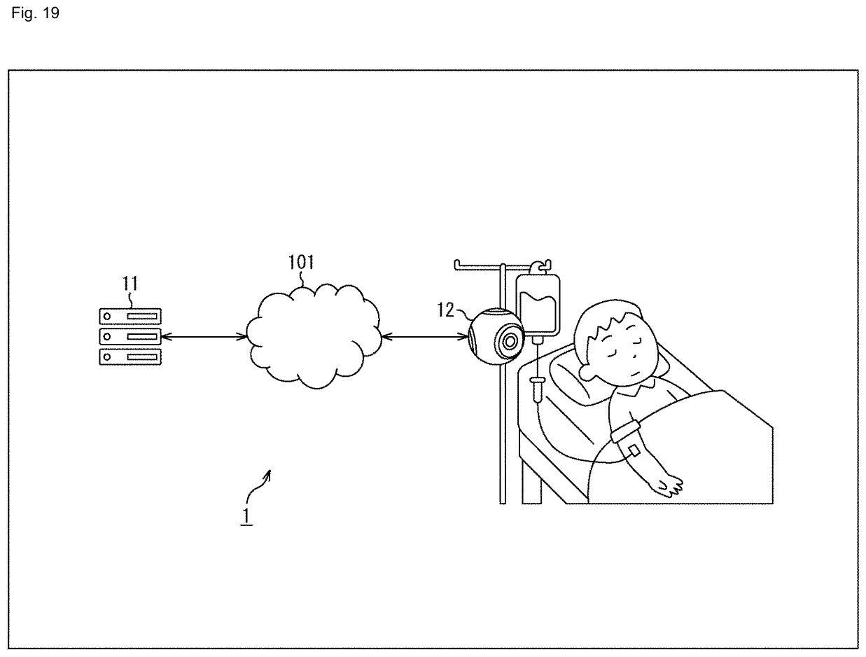

FIG. 19 is a diagram illustrating another configuration example of the respiratory monitoring system 1.

In the example of FIG. 1, the information processing device 11 is installed in a medical facility. However, as illustrated in FIG. 19, a server on cloud may function as an information processing device 11. Communication is performed between the information processing device 11 installed outside a medical facility and an RGB-D camera 12 installed inside the medical facility via a network 101 such as the Internet. Although not illustrated, a monitor 13 is installed, for example, in the medical facility.

For example, in the RGB-D camera 12, respiratory waveform data as described above is generated based on RGB images and Depth images, and is transmitted to the information processing device 11. At this time, the RGB image(s) and the Depth image(s) may be transmitted to the information processing device 11, or may not be transmitted.

The information processing device 11 performs the above-described respiratory monitoring processing based on the respiratory waveform data transmitted from the RGB-D camera 12. In this way, the RGB-D camera 12 functions as an edge device and only the respiratory waveform data is transmitted to the information processing device 11, making it possible to keep images in which a patient appears inside the medical facility. This makes it possible to realize a respiratory monitoring system that respects patient privacy.

In addition, by performing high-load signal processing such as analysis of respiratory waveform data and classification into a respiration pattern in the information processing device 11, the processing load of the RGB-D camera 12 can be reduced, making it possible to achieve miniaturization and energy saving of the RGB-D camera 12.

### <Configuration of RGB-D Camera 12>

As the sensors of the RGB-D camera 12, an RGB-D coaxial sensor can be used in which a sensor for RGB images and a sensor for Depth images are arranged to serve as coaxial sensors. By using the RGB-D coaxial sensors, the housing of the RGB-D camera 12 can be made smaller and less expensive. Furthermore, there is no need to align the sensor for RGB images and the sensor for Depth images.

The RGB-D camera 12 may be equipped with a small monitor. This makes it possible to realize a small respiratory monitoring camera equipped with RGB-D coaxial sensors. By using the RGB-D coaxial sensors to achieve miniaturization and energy saving, the RGB-D camera 12 can be configured as a battery-powered device.

This eliminates the need for a power cable and the like, and allows for greater freedom in installation of the RGB-D camera 12. In addition, providing the RGB-D camera 12 with a display unit 13A that is a small monitor makes it possible to check the respiratory waveforms at the location where the RGB-D camera 12 is installed.

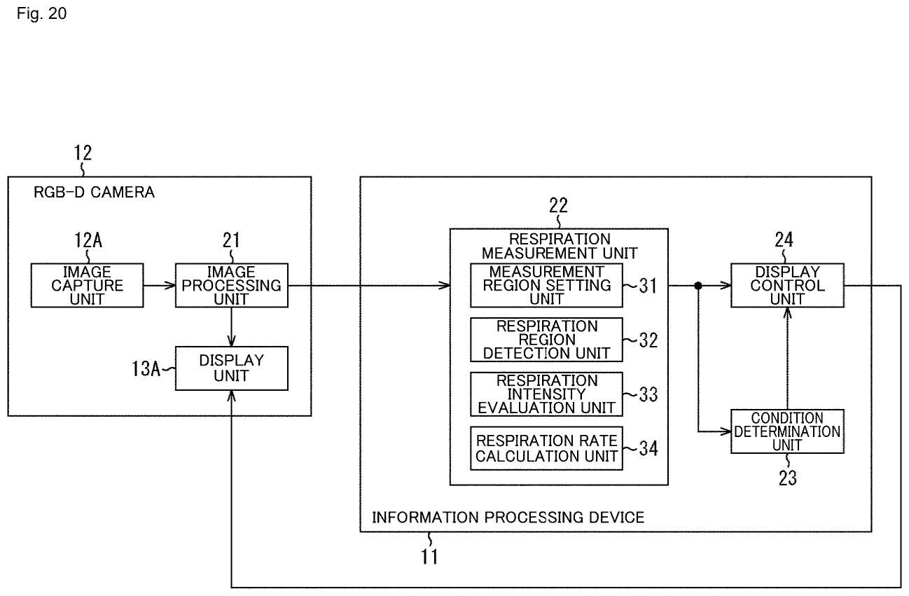

A configuration example of the RGB-D camera 12 that functions as an edge device and is equipped with a small monitor is illustrated in FIG. 20. In the example of FIG. 20, an image processing unit 21 and the display unit 13A are provided in the RGB-D camera 12.

The image processing unit 21 of the RGB-D camera 12 extracts image data of an ROI of each of an RGB image and a Depth image, which are captured by an image capture unit 12A, and performs image processing on the ROIs to analyze their image feature amounts. The image processing unit 21 outputs the respiratory waveform data, which is the analysis results of the image feature amounts of the ROIs, to a respiration measurement unit 22.

The display unit 13A of the RGB-D camera 12 displays a monitoring screen under the control of a display control unit 24 of the information processing device 11.

The RGB-D camera 12 may be connected to an external device such as a smartphone to enable remote control.

### Others

### Modification Example of Initial Setting of ROI

The initial setting of the ROI has been described which is performed manually by a medical staff member. However, it may be performed by automatic ROI search processing.

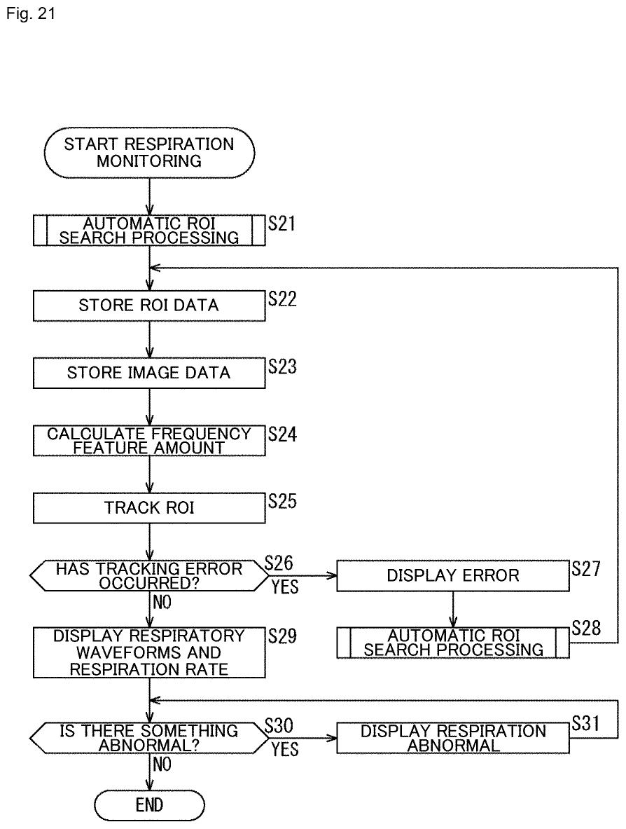

FIG. 21 is a flowchart illustrating respiratory monitoring processing for initial setting of the ROI automatically performed.

The processing illustrated in FIG. 21 is similar to the processing illustrated in FIG. 8, except that the same processing as the automatic ROI search processing described with reference to FIG. 11 is performed in step S21. Specifically, the initial setting of the ROI is automatically performed by the automatic ROI search processing, and then the processing of step S22 and subsequent steps are performed.

Processing of steps S22 to S31 is the same as processing of steps S1 to S10 of FIG. 8. When a tracking error of the automatically set ROI occurs, the automatic ROI search processing is performed in step S28.

### Modification Example of Tracking of ROI

An ROI has been described which is tracked by movement estimation such as the Optical Flow method. However, an ROI may be tracked by another algorithm based on image feature amounts. For example, an ROI can be tracked using template matching.

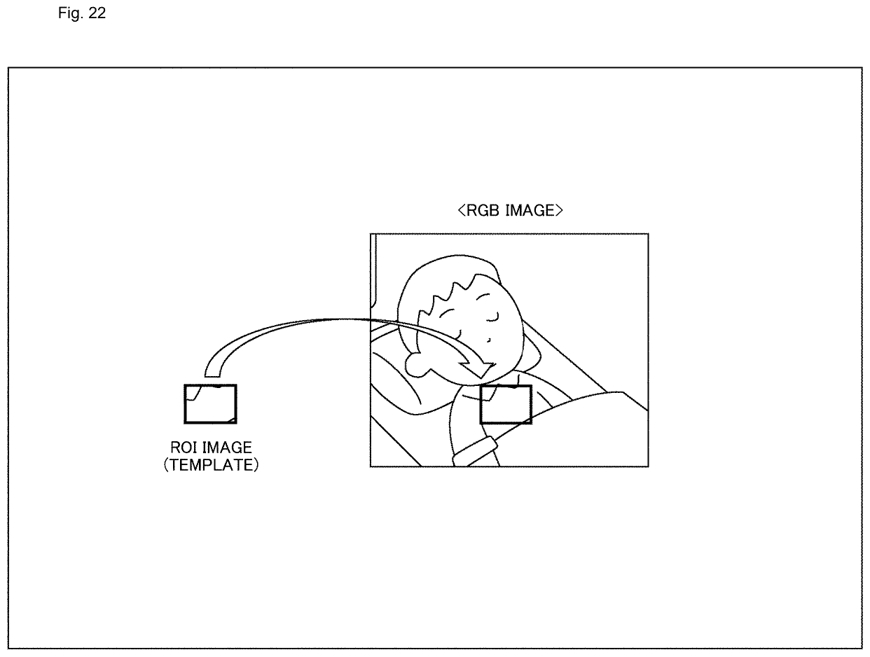

FIG. 22 is a diagram illustrating an example of tracking of an ROI using template matching.

An ROI image, which is an image of the ROI in the entire RGB image, is used as a template, and matching of the ROI image with each region in the RGB image is performed as indicated by an arrow in FIG. 22. For example, the ROI is tracked using template matching so as to detect the region with the highest similarity in image feature amounts.

Tracking of an ROI using template matching is performed, for example, as follows.
Image feature amounts of the ROI are stored in a memory, and the ROI is tracked for each frame.
When a tracking error occurs due to, for example, occlusion, tracking will be attempted for a specified period of time (a period of time during which failure to detect respiration will not be a significant clinical problem, such as 10 seconds).
When tracking is not possible even after the specified period of time has elapsed, an error is detected, and then an automatic search for the ROI is performed.

Tracking of an ROI based on image feature amounts using the Optical Flow method, template matching, or the like may be performed using a Depth image(s). The ROI may be tracked using image feature amounts of both an RGB image(s) and a Depth image(s). In other words, it is possible to track the ROI using the image feature amounts of at least one of the RGB image(s) and the Depth image(s).

The ROI may be tracked using the frequency feature amount of at least one of the RGB image(s) and the Depth image(s).

Each processing having been described which is performed using an RGB image(s) may be performed using an IR image(s) instead of the RGB image(s). When there is no lighting, such as at night, it can be difficult to observe the subject using an RGB image(s). Using an IR image(s) makes it possible to perform various types of processing such as tracking an ROI and analyzing image feature amounts.

Since IR light is used to capture a Depth image, the RGB-D camera 12 can also capture an IR image at the same time as capturing the Depth image. The IR image captured together with the Depth image is used instead of an RGB image.

### Configuration Example of Computer

The series of processing described above can be executed by hardware or software. When executing the series of processing by software, a program that configures the software is installed from a program recording medium onto either a computer incorporated in dedicated hardware or a general-purpose personal computer.

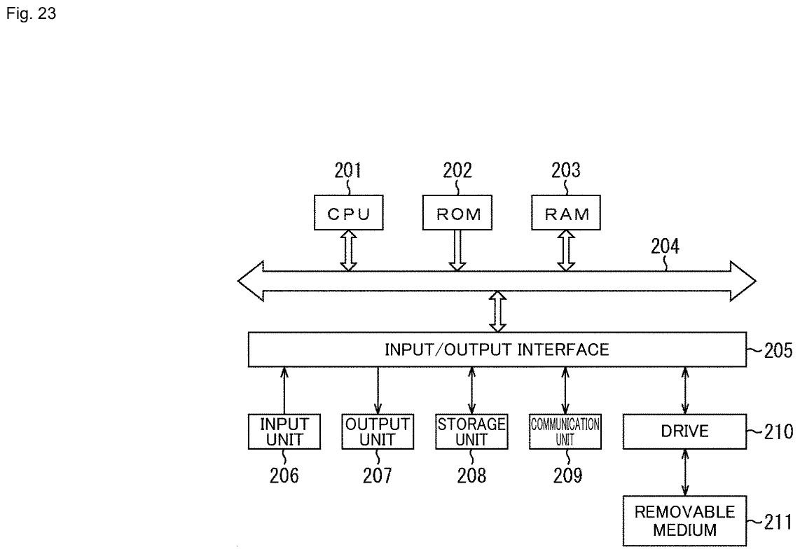

FIG. 23 is a block diagram illustrating a configuration example of hardware of a computer that executes the series of processing described above by a program. The information processing device 11 is also configured with the computer having the configuration illustrated in FIG. 23.

A Central Processing Unit (CPU) 201, a Read Only Memory (ROM) 202, and a Random Access Memory (RAM) 203 are connected to one another via a bus 204.

An input/output interface 205 is further connected to the bus 204. An input unit 206 including a keyboard and a mouse and an output unit 207 including a display and a speaker are connected to the input/output interface 205. In addition, a storage unit 208 including a hard disk, a non-volatile memory, and the like, a communication unit 209 including a network interface and the like, and a drive 210 that drives a removable medium 211 are connected to the input/output interface 205.

In the computer configured as described above, for example, the CPU 201 loads a program stored in the storage unit 208 into the RAM 203 via the input/output interface 205 and the bus 204 and executes the program, to perform the above-described series of processing.

The program executed by the CPU 201 is recorded on, for example, the removable medium 211 or is provided via a wired or wireless transfer medium such as a local area network, the Internet, or digital broadcast to be installed in the storage unit 208.

The program executed by the computer may be a program that performs a plurality of types of processing in time series in the order described herein or may be a program that performs processing in parallel or at a necessary timing such as when invoked.

A system as used herein means a collection of a plurality of constituent elements (including devices and modules (components)) regardless of whether all the constituent elements are contained in the same casing. Accordingly, a plurality of devices accommodated in separate casings and connected via a network and one device in which a plurality of modules are accommodated in one casing both constitute systems.

The advantageous effects described herein are merely examples and are not intended as limiting, and other advantageous effects may be acquired.

The embodiments of the present technology are not limited to the above-described embodiments, and various changes can be made without departing from the scope and spirit of the present technology.

For example, the present technology may be configured as cloud computing in which a single function is cooperatively processed in a distributed manner via a network.

In addition, each step described in the flowchart discussed above can be executed by a single device, or executed by a plurality of devices in a distributed manner.

Furthermore, when a single step includes a plurality of types of processing, the plurality of types of processing included in the single step can be performed by a single device, or in a distributed manner by a plurality of devices.

### Combination Example of Configurations

The present technology can be configured as follows.

(1)

A monitoring system including:
an image processing unit that acquires an RGB image obtained by capturing an image of a patient and a Depth image indicating Depth values relation to certain locations on the patient; and
a measurement unit that tracks, based on the RGB image, a measurement region set in a respiration region of the patient, and, when a tracking error of the measurement region occurs, resets the measurement region, based on the Depth image.
   (2)

The monitoring system according to (1), wherein the image processing unit acquires the RGB image and the Depth image that are captured by an RGB-D camera.

(3)

The monitoring system according to (1) or (2), wherein the measurement unit tracks the measurement region by using the Depth image together with the RGB image.

(4)

The monitoring system according to (3), wherein the measurement unit tracks the measurement region, based on an image feature amount of the measurement region in the RGB image and an image feature amount of the measurement region in the Depth image.

(5)

The monitoring system according to any one of (1) to (4), wherein the measurement unit resets the measurement region, based on a frequency feature amount of the measurement region in the Depth image.

(6)

The monitoring system according to (5), wherein the measurement unit calculates a frequency feature amount of the measurement region by analyzing waveform data indicating changes in Depth values.

(7)

The monitoring system according to (6), wherein when resetting the measurement region, the measurement unit performs selection of regions that are to be candidates for the measurement region, based on respective Depth values.

(8)

The monitoring system according to (7), wherein the measurement unit excludes, so as to perform the selection, at least any of: a region where a ratio of pixels having undetected Depth values is greater than a threshold value, a region where a distance to a subject is outside a predetermined range, and a region where a coefficient of variation of the Depth values is greater than a threshold value.

(6)

The monitoring system according to (7) or (8), wherein the measurement unit determines, as the respiration region, a region having a frequency feature amount in which a coefficient of determination for a regression curve is smaller than a threshold value.

(10)

The monitoring system according to (9), wherein the measurement unit calculates a respiration intensity of the respiration region, based on a frequency feature amount in a predetermined frequency range including a peak frequency, and selects the respiration region as the measurement region.

(11)

The monitoring system according to any one of (1) to (10), wherein the image processing unit generates, by analyzing the measurement region in the RGB image, waveform data indicating changes in luminance values and waveform data indicating changes in amount of movement of a subject in a planar direction, and
generates, by analyzing the measurement region in the Depth image, waveform data indicating changes in amount of movement in a depth direction.
   (12)

The monitoring system according to (11), wherein
the measurement unit calculates a frequency feature amount of the measurement region by analyzing at least any of the waveform data,
the monitoring system further comprising a determination unit that determines a respiratory condition of the patient, based on the frequency feature amount of the measurement region.
   (13)

The monitoring system according to (12), further including a display control unit that causes a respiratory waveform to be displayed, based on at least any of the waveform data.

(14)

The monitoring system according to (13), wherein
the measurement unit calculates a respiration rate, based on a peak frequency in
the frequency feature amount of the measurement region, and
the display control unit causes the respiration rate of the patient, together with the respiratory waveform, to be displayed.
   (15)

A monitoring method including: by a monitoring system,
acquiring an RGB image obtained by capturing an image of a patient and a Depth image indicating Depth values relative to certain locations on the patient;
tracking, based on the RGB image, a measurement region set in a respiration region of the patient; and
resetting, when a tracking error of the measurement region occurs, the measurement region, based on the Depth image.
   (16)

A program causing a computer to execute:
acquiring an RGB image obtained by capturing an image of a patient and a Depth image indicating Depth values relative to certain locations on the patient;
tracking, based on the RGB image, a measurement region set in a respiration region of the patient; and
resetting, when a tracking error of the measurement region occurs, the measurement region, based on the Depth image.

### REFERENCE SIGNS LIST

1 Respiratory monitoring system
11 Information processing device
12 RGB-D camera
13 Monitor
21 Image processing unit
22 Respiration measurement unit
23 Condition determination unit
24 Display control unit
31 Measurement region setting unit
32 Respiration region detection unit
33 Respiration intensity evaluation unit
34 Respiration rate calculation unit

1. A monitoring system comprising:
an image processing unit that acquires an RGB image obtained by capturing an image of a patient and a Depth image indicating Depth values relative to certain locations on the patient; and
a measurement unit that tracks, based on the RGB image, a measurement region set in a respiration region of the patient, and, when a tracking error of the measurement region occurs, resets the measurement region, based on the Depth image.

2. The monitoring system according to claim 1, wherein the image processing unit acquires the RGB image and the Depth image that are captured by an RGB-D camera.

3. The monitoring system according to claim 1, wherein the measurement unit tracks the measurement region by using the Depth image together with the RGB image.

4. The monitoring system according to claim 3, wherein the measurement unit tracks the measurement region, based on an image feature amount of the measurement region in the RGB image and an image feature amount of the measurement region in the Depth image.

5. The monitoring system according to claim 1, wherein the measurement unit resets the measurement region, based on a frequency feature amount of the measurement region in the Depth image.

6. The monitoring system according to claim 5, wherein the measurement unit calculates a frequency feature amount of the measurement region by analyzing waveform data indicating changes in Depth values.

7. The monitoring system according to claim 6, wherein when resetting the measurement region, the measurement unit performs selection of regions that are to be candidates for the measurement region, based on respective Depth values.

8. The monitoring system according to claim 7, wherein the measurement unit excludes, so as to perform the selection, at least any of: a region where a ratio of pixels having undetected Depth values is greater than a threshold value, a region where a distance to a subject is outside a predetermined range, and a region where a coefficient of variation of the Depth values is greater than a threshold value.

9. The monitoring system according to claim 7, wherein the measurement unit determines, as the respiration region, a region having a frequency feature amount in which a coefficient of determination for a regression curve is smaller than a threshold value.

10. The monitoring system according to claim 9, wherein the measurement unit calculates a respiration intensity of the respiration region, based on a frequency feature amount in a predetermined frequency range including a peak frequency, and selects the respiration region as the measurement region.

11. The monitoring system according to claim 1, wherein the image processing unit generates, by analyzing the measurement region in the RGB image, waveform data indicating changes in luminance values and waveform data indicating changes in amount of movement of a subject in a planar direction, and
generates, by analyzing the measurement region in the Depth image, waveform data indicating changes in amount of movement in a depth direction.

12. The monitoring system according to claim 11, wherein
the measurement unit calculates a frequency feature amount of the measurement region by analyzing at least any of the waveform data,
the monitoring system further comprising a determination unit that determines a respiratory condition of the patient, based on the frequency feature amount of the measurement region.

13. The monitoring system according to claim 12, further comprising a display control unit that causes a respiratory waveform to be displayed, based on at least any of the waveform data.

14. The monitoring system according to claim 13, wherein
the measurement unit calculates a respiration rate, based on a peak frequency in the frequency feature amount of the measurement region, and
the display control unit causes the respiration rate of the patient, together with the respiratory waveform, to be displayed.

15. A monitoring method comprising: by a monitoring system,
acquiring an RGB image obtained by capturing an image of a patient and a Depth image indicating Depth values relative to certain locations on the patient;
tracking, based on the RGB image, a measurement region set in a respiration region of the patient; and
resetting, when a tracking error of the measurement region occurs, the measurement region, based on the Depth image.

16. A program causing a computer to execute:
acquiring an RGB image obtained by capturing an image of a patient and a Depth image indicating Depth values relative to certain locations on the patient;
tracking, based on the RGB image, a measurement region set in a respiration region of the patient; and
resetting, when a tracking error of the measurement region occurs, the measurement region, based on the Depth image.
